(19) United States
(12) Patent Application Publication
(10) Pub. No.: US 2023/0070800 A1
(43) Pub. Date: Mar. 9, 2023

(54) CEREBRAL EMBOLIC PROTECTION DURING OPEN HEART SURGERY

(71) Applicant: ENCOMPASS TECHNOLOGIES, INC., San Clemente, CA (US)

(72) Inventors: Michael Jones, San Clemente, CA (US); George Wallace, San Clemente, CA (US); Brady Haug, San Clemente, CA (US)

(21) Appl. No.: 18/055,307

(22) Filed: Nov. 14, 2022

Related U.S. Application Data
(63) Continuation-in-part of application No. 16/996,657, filed on Aug. 18, 2020.
Application No. 16/996,657 is a continuation-in-part of application No. 16/868,076, filed on May 6, 2020.
(60) Provisional application No. 62/888,897, filed on Aug. 19, 2019.
(60) Provisional application No. 63/279,816, filed on Nov. 16, 2021.

(51) Int. Cl. A61F 2/01; A61F 2/24

(52) U.S. Cl. CPC A61F 2/01; A61F 2/2436; A61F 2002/016

(57) ABSTRACT

An intravascular filter for blocking passage of embolic debris into the cerebral and aortic circulation upon removal of the cross clamp from the aorta during open heart surgery. The filter comprises a self expandable tubular wire frame, having a proximal end, a distal end and a lumen defined within a tubular sidewall. A tubular porous membrane is carried by the sidewall and extends across the proximal or distal end, so that debris entering the other end can be captured within the lumen, the membrane having a distribution of pore sizes. A control wire extends proximally from the filter. In use, the aorta may be cross clamped over the control wire or over the filter. Following removal of the cross clamp, blood is allowed to perfuse through the membrane in the direction of the descending aorta while retaining embolic debris therein for subsequent removal, and the filter and debris may be proximally retracted using the control wire.

### CROSS-REFERENCE TO RELATED APPLICATIONS

This application is a continuation-in-part of U.S. application Ser. No. 16/996,657, filed Aug. 18, 2020, which is a continuation-in-part of U.S. application Ser. No. 16/868,076, filed May 6, 2020, which claims the priority benefit under 35 U.S.C. § 119(e) of U.S. Provisional Application No. 62/888,897, filed Aug. 19, 2019, and this application claims the priority benefit under 35 U.S.C. § 119(e) of U.S. Provisional Application No. 63/279,816, filed Nov. 16, 2021, the entireties of each of which is hereby incorporated by reference herein.

### BACKGROUND

A wide variety of procedures can be done on the heart, including heart transplant, valve repair, valve replacement, and others. Many are conducted through an open surgical approach, with a growing number being replaced by trans vascular catheter based procedures.

In all of the foregoing procedures, there is a risk of dislodging calcifications or other debris, which travels (embolizes) up the ascending aorta, enters the cerebral circulation and cause a stroke.

During an open surgical heart repair, the heart is arrested and the patient's blood is perfused through an extracorporeal circuit to an external mechanical heart lung machine. This requires among other things clamping the aorta with a cross clamp while blood is diverted into the extracorporeal circuit. At the end of the procedure, when the patient is taken off the heart-lung machine and native vasculature is repaired, the cross clamp is removed from the aorta to allow normal blood flow to resume.

A well understood consequence of this procedure is the release of embolic debris at the time of removing the cross clamp from the aorta. That debris can enter the cerebral circulation and cause a stroke.

Thus, there remains a need for a technology for minimizing the risk of stroke as a result of embolic debris released upon removal of the cross clamp from the aorta, in surgical heart repair.

### SUMMARY

There is provided in accordance with one aspect of the present invention an intravascular filter for blocking passage of selected sizes of debris. The filter comprises a self expandable tubular wire frame, having a first end, a second end and a tubular sidewall defining a central lumen; and a tubular porous membrane carried by the sidewall, the membrane having a distribution of pore sizes. A second group of pores has pores with a maximum average dimension of no more than about 25 microns and a first group of pores has pores with a maximum average dimension of at least about 50 microns but no more than about 100 microns or 80 microns, and the prevalence of pores in the second group is at least three times the prevalence of pores in the first group. A porous membrane extends across the second end, so that debris entering the first end can be captured within the central lumen.

The second group of pores may block particles greater than about 120 microns, or may block particles greater than about 100 microns. The second group of pores may block particles greater than about 80 microns. The prevalence of pores in the first group may be at least four times the prevalence of pores in the second group.

A pressure drop across the filter may be less than about 10 mm Hg at physiologic flow rates, or may be less than about 5 mm Hg at physiologic flow rates.

The sum of the area of all of the pores may be at least about 30% of the surface area of the membrane, or may be at least about 35% of the surface area of the membrane. In some embodiments, the tubular porous membrane may comprise electrospun fibers.

There is also provided an embolic protection system, comprising a self expandable frame having a proximal end and a distal end; and a filter membrane supported by the tubular body, surrounding the tubular body and covering the distal end. A transition is provided between the frame and a control wire extending proximally from the transition. The transition may comprise a first set of wires extending proximally from the frame to a first set of welds, and a second, smaller set of wires extending proximally from the welds, and the welds are axially displaced from each other.

The embolic protection system may further comprise a tubular delivery catheter, and the frame is carried in a reduced cross-sectional configuration within the delivery catheter. The delivery catheter may have an outer diameter of less than 14 F, such as down to 8 Fr or less. In some embodiments, the tubular porous membrane may comprise electrospun fibers. The filter membrane may have a distribution of pore sizes, wherein a first group of pores has pores with a maximum dimension of no more than about 25 microns and a second group of pores has pores with a maximum dimension of at least about 50 microns, and the prevalence of pores in the first group is at least three times the prevalence of pores in the second group

There is also provided a method of protecting the cerebral vasculature from embolic debris, following removal of a clamp on the aorta to facilitate open heart surgery. The method includes providing an embolic protection delivery catheter having a tubular restraint for restraining an embolic protection filter in a reduced profile configuration. The filter may have a self expandable wire frame, a filter membrane carried by the frame and a proximal and distal radiopaque markers. The filter membrane may cover at least an end of the frame.

The filter is advanced distally through an access sheath into the aorta; the restraint is retracted to expose the filter and permit the frame to radially expand, leaving a control wire extending proximally from the filter. The aorta may then be cross clamped. In some embodiments, the cross clamping step may comprise cross clamping the aorta over the embolic protection filter. The cross clamping step may comprise cross clamping the aorta over the control wire. The method may further comprise removing the cross clamp from the aorta and allowing blood to perfuse through the membrane in the direction of the descending aorta while retaining embolic debris therein for subsequent removal. The embolic protection delivery catheter may be delivered to the aorta via i) direct placement of the filter during open cardiac surgery, ii) through an aortic cannula hole, iii) through a left radial artery or left subclavian artery, or iv) trans-apically.

### BRIEF DESCRIPTION OF THE DRAWINGS

FIG. 1 is a side elevational view of a filter frame in accordance with the present invention, with the filter membrane removed.

FIG. 2A is a side elevational view of an alternate frame having a filter membrane thereon.

FIG. 2B is a perspective view of the frame from the filter of FIG. 2A.

FIG. 3 is a histogram showing the pore size distribution of a filter membrane.

FIG. 4 shows the radial force as a function of diameter for a filter frame.

FIGS. 5A through 5C show different filter configurations.

FIGS. 6A through 6C show direct placement of the filter during open cardiac surgery.

FIGS. 7A through 7D show placement of the filter through the aortic cannula hole.

FIGS. 8A through 8C show filter deployment via the left subclavian or left radial artery, with two different filter configurations.

FIGS. 9A through 9C show filter deployment via transapical access.

### DETAILED DESCRIPTION

A filter comprising a frame and filter membrane are disclosed for coverage of the great vessels leading to the brain during coronary surgical procedures, including surgical aortic valve repair or replacement, mitral valve repair or replacement, coronary artery bypass surgery and other surgical procedures that require a heart lung machine to perfuse the body with oxygenated blood, manipulation of the heart, or cross clamping of the aorta. The shorthand term ‘filter’ is intended to refer to the frame and filter membrane combination disclosed herein which has both filtration functions and debris deflection functions. Terms like debris and particles are used interchangeably herein and are not intended to convey separate meanings.

The filter frame is thus in the form of a tubular body having a central lumen extending between a proximal opening and a distal opening. In implementations intended for insertion via the heart or directly into the ascending aorta, the proximal opening preferably remains unobstructed to enable entry of embolic debris into the central lumen. The distal opening of the frame may be covered with filter membrane to enclose the central lumen, allowing blood to perfuse through the membrane in the direction of the descending aorta while retaining embolic debris therein for subsequent removal. Alternatively, the distal opening may remain unobstructed, allowing debris to travel downstream into the peripheral vasculature.

The filter will filter oxygenated blood that is pumped from the heart lung machine through the aortic cannula and into the aortic arch during bypass. Any debris generated from the heart lung machine, manipulation of the heart, or removing the cross clamp from the aorta will be deflected away from the great vessels leading to the brain, and/or collected in the filter and removed, after the procedure is complete.

The device of the present invention is configured to block debris above one or two or three or more predetermined threshold sizes from entering the cerebral circulation. Some blocked debris may become entrapped in the membrane, while other blocked debris will be deflected and travel downstream through the aorta. Preferably, all debris passing through the filter will have a particle size below about 140μ, at least about 80% or about 90% or about 95% below 80μ and very few below about 20μ.

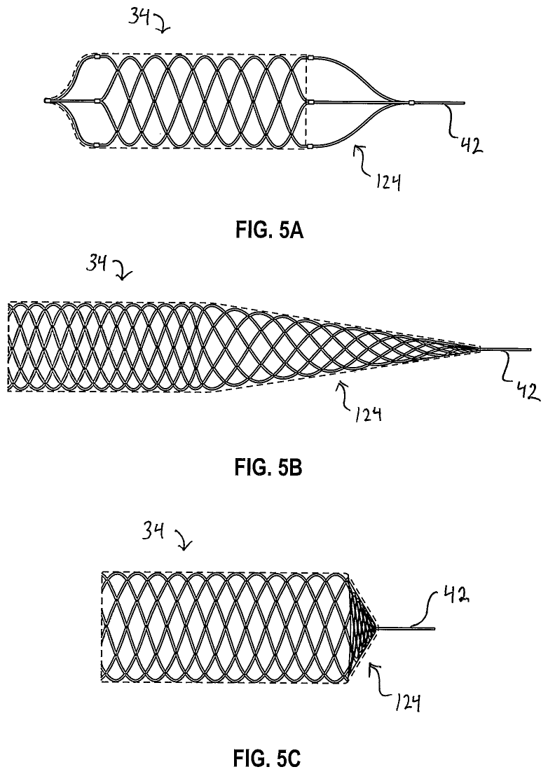

Certain configurations such as those shown in FIGS. 5, 6 and 8 may have a closed downstream end to retain any particulate emboli within the closed end of the filter.

Certain characteristics of the design include 1) Minimal imaging is required because fluoroscopy is not required for surgical placement; 2) No TAVR devices or other accessories are anticipated to go through the filter once placed in the aorta; 3) The filter is in place during the potentially major debris events related to aortic cross-clamping and unclamping to allow restoration of blood flow; 4) the filter is compatible with the aortic cannula for perfusion of the brain.

The delivery system is preferably less than 12F in diameter (OD) and capable of delivering the filter to the ascending aorta and potentially over the aortic arch to the descending aorta. One implementation of a 0.015 inch, five wire frame (described below) fits within a 12Fr ID delivery system and expands out to about 32 mm for an 8:1 expansion ratio. A frame configured for the contralateral application is the same deployed OD (32 mm) but collapses to 9Fr for deployment for a 10.6:1 expansion ratio. Expansion ratios of at least about 6:1 or at least about 7:1 or at least about 8:1 may be provided for the filter of the present invention.

The filter may be deployed by maintaining the axial position of the collapsed filter with a core wire and proximally retracting an outer tubular restraint to expose the filter (like a self expanding stent) and allow it to expand within the aorta. Expansion is preferably up to a diameter of at least about 30 mm or at least about 35 mm or at least about 40 mm.

The delivery system is capable of accessing the vascular system from the left radial artery, the left subclavian artery, trans-apically, through the hole for the arterial cannula access or through the arterial cannula without the need for fluoroscopy. Access may be achieved under direct vision, or transesophageal ultrasound to position and deploy the filter in the aortic arch, while the frame/filter is tethered to a retrieval wire that is used to retrieve the frame/filter post procedure. Ultrasound visualization (e.g., TEE) of filter deployment in the aortic arch may desirably be further enhanced by the addition of at least one of two or four or more ultrasound markers to the wire frame.

The frame may comprise a small wire diameter (e.g., no more than about 0.02 inches, such as about 0.015 inches, or no more than about 0.009 inches, such as about 0.006 inches, or about 0.009 inches) for the frame construction. In some embodiments, the wire is nitinol and is 0.006 inches in diameter. In some embodiments, the wire is nitinol and is 0.009 inches in diameter. The small wire diameter allows for minimizing delivery profile. This small diameter may result in relatively low device stability, however, due to wire anchoring and the fact that catheters will not be passed through the filter, sufficient stability can be achieved. Wire anchoring is enhanced by radially enlarged proximal and distal landing zones on the frame as well as the length of the frame extending along the arch as well as the axial integrity of the retrieval wire under tension or compression depending upon the location of the vascular access. The filter membrane may be have pores with maximum average size of no more than about 100 microns or no more than about 80 microns with graduated pore sizes below 80 microns. This has demonstrated significant cerebral protection without impacting blood flow to the brain.

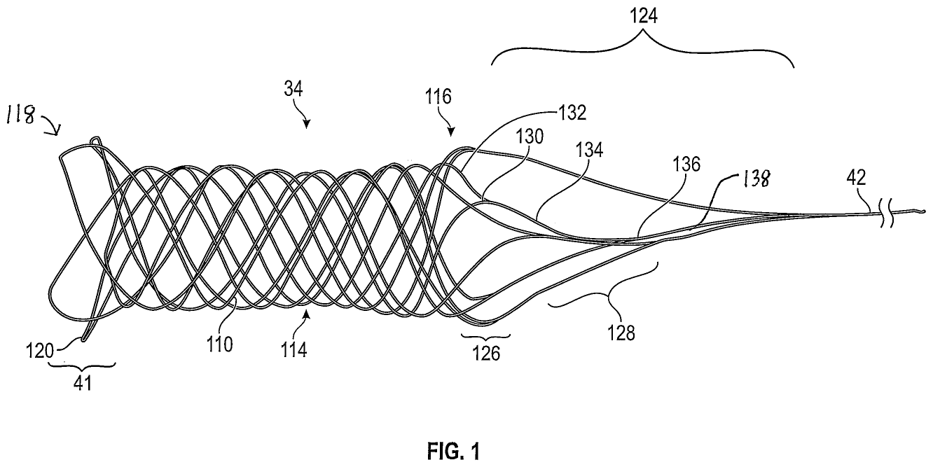

Referring to FIG. 1, one implementation of a filter frame 34 (with the filter membrane omitted) is illustrated. The frame 34 is shown in an unconstrained expansion, having a plurality of sidewall wires 110 forming a tubular sidewall 114 extending between a proximal end 116 and a distal end 118. The sidewall 114 may include a series within the range of from about three to about 10 and in one implementation five wires 110 which in one implementation are 0.0155″ (inches), or 0.006″, or 0.009″ nitinol wires. The wires 110 may have straight lengths before winding within the range of from about 15″ to about 40″, generally within the range of from about 20″ to about 35″ and in one example are about 27″ in length. While nitinol wire is preferred, cobalt chrome wire is also heat treatable and while not superelastic is used in self-expanding stents.

Each wire 110 extends helically distally along the sidewall to the distal end 118 where it folds back to form an apex 120 and extends helically proximally so both ends of the wire are in a proximal location on the frame 34. Alternatively, the distal ends of wires can be free ends, or welded to one or more adjacent wire ends.

In the illustrated implementation, the wires 110 are woven over a mandrel with distal loops 120 inclining radially outwardly (bend angle is about 125 degrees) in the landing zone 41 creating a trumpet flare at the distal end 118 having an OD of at least about 35 mm or 40 mm or in one implementation about 45 mm in an unconstrained expansion. A smaller generally cylindrical diameter along the mid-section may be no more than about 35 mm or 32 mm in an unconstrained expansion. A landing zone in the form of a radially enlarged proximal bulge adjacent the proximal end 116 may have an OD of at least about 35 mm or at least about 40 mm. Alternatively, the unconstrained expansion may produce a substantially constant diameter throughout the length of the frame, or either the proximal enlargement or the distal enlargement may be used without the other, depending upon the desired performance and intended vessel size.

The larger diameter (trumpet or flared) portion adjacent the distal end 118 may help with stabilization and reduce the risk of axial migration within the aorta. The proximal bulge may help force the frame out to seat against the wall of the descending aorta and help with anchoring.

The illustrated embodiment produces five distal apexes 120 and ten proximal ends because of the use of five folded wires. Three wires may be useful in small vessel applications, and no more than about 7 or 10 wires may be used but with a corresponding increase in crossing profile. The proximal wire ends are off set circumferentially so that they extend proximally from the tubular sidewall all within an arc of no more than about 270 degrees or 220 degrees or less of the circumference of the tubular sidewall.

The proximal end of each wire is connected to an adjacent wire in a series of axially spaced apart junctions distributed over a transition zone 124 that may have an axial length of at least about 1 cm, preferably at least about 2 cm and in some embodiments at least about 2.5 cm or 3 cm. Axially offsetting the welds optimizes the collapsed crossing profile by avoiding welds stacking up in a common transverse plane.

The wires in the transition zone ramp the OD down from the expanded diameter of the proximal end 116 of the frame (e,g, at least about 30 mm or 40 mm) down to a single control wire 42. This is accomplished with a series of wire side welds with a first weld zone 126 to transition from 10 wires down to five. In a second, proximal weld zone 128 wires are transitioned from five down to three and then three down to one resulting in all wires funneling down in a proximal direction into a single (e.g., 0155″) control wire 42 that runs the length of the delivery catheter, through the handle and exits proximally at the hemostatic valve.

For example a first weld 130 in the first weld zone 126 joins a proximal end of a first wire 132 to a side of a second wire 134. Five such welds are shown in the first weld zone 126. Second wire 134, for example continues proximally to the second weld zone 128 where an end weld 136 joins it to a third wire 138. Three such wires 138 continue proximally to the control wire 42.

This results in one implementation in an un-restrained length of about 9 cm for the filter. The actual length in the aorta will be determined by the vessel diameter over the 9 cm length (smaller diameters result in elongation). The length of this frame when retained inside the delivery catheter in one implementation is about 24.3 cm. The collapsed length is generally at least about 2 times or at least about 2.5 times the length upon unconstrained expansion. The woven wires may be spaced about 72 degrees apart on the mandrel and heat set for 20-26 minutes at about 920 degrees (to maintain outside diameter and spacing between the wires).

Each wire 110 makes no more than 10, typically no more than six and in some implementations no more than four complete revolutions about the longitudinal axis of the frame. This results in axial lengthening of at least about 100% (doubling) or 150% or 175% or more in response to radial compression upon loading into the 14F tubular body 31.

In a frame made from 0.015″ wire, the percent coverage of the expanded side wall by the sum of the radially outwardly facing wire area may be within the range of from about 5% to about 15% and in one implementation is about 10%.

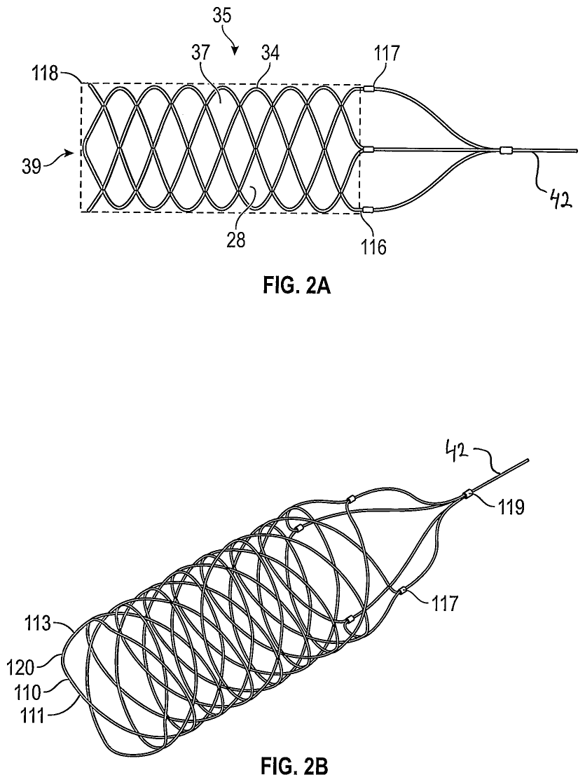

Referring to FIGS. 2A and 2B, a filter 35 comprises a frame 34 supporting a filter membrane 37. The filter membrane 37 may extend at least about 50% or at least about 70% of the way around the circumference, and preferably forms a complete tubular layer around a central cavity enclosed by the frame 34, and also extends across a closed end 39. Thus while the proximal end of the filter 35 is open to receive blood and debris, the distal end is closed to debris as discussed herein, but permits blood flow downstream through the closed end.

A distal apex 120 on a wire 110 is illustrated in additional detail in FIG. 2B. The wire 110 is folded back upon itself at apex 120 to provide a first strut 111 extending helically in a proximal direction on a first side of apex 120 and a second strut 113 on a second side of apex 120, also extending proximally in a helical pattern.

In any of the embodiments disclosed herein, at least one or two or three or more radiopaque markers may be provided if desired for fluoroscopic visualization. For example, three or four or more radiopaque markers 117 may be provided at the proximal end 116 of the frame 34. Optionally, an additional radiopaque marker 119 may be provided such as at the transition to a single control wire 42.

The control wire 42 extends from the frame 34 proximally to a filter control on the proximal end of the catheter. Proximal motion of an outer tubular constraint relative to the control wire 42 and a pusher will retract the tubular body to uncover the filter leaving it unconstrained. This allows the frame 34 to self expand into, for example, a tubular configuration, having a diameter of at least about 20 mm or 25 mm to about 30 mm or 35 mm or more, and to support the membrane 36 against the wall of the aorta.

Once the frame is heat set, it may be tumbled and electropolished for a rough surface. The frame may then be coated such as by dip coating in polyurethane to form fixed position crossing points where adjacent crossing filaments can pivot about the crossing point but are restrained from sliding axially along either wire.

Coating the frame prior to applying the electro spun coating (membrane) thus holds the wire positions relative to other wires throughout the range of expansion and keeps the wires from sliding relative to one another. This creates a consistent hinge out of each wire crossing point. The coating also provides a better surface for the electro spun coating (membrane) to adhere to compared to the bare metal frame. Both of these features add to filter durability. Adding additional electro spun material (more passes of the spinneret) to the proximal and distal end sections of the frame, where the diameter is at its largest (40-45 mm), increases the adherence of the coating (membrane) to the frame. These areas will see the highest stress points to the filter/frame during the procedure.

The coated frame may then be loaded on a mandrel and electro spun with a polyurethane (Tecothane or other) dispersion at a shore hardness that yields a cell covering that can expand and contract with frame expansion and contraction. The material is applied to the frame through an electro-spinning process onto the rotating mandrel that results in a covering (membrane) that yields a distribution of different sized openings or pores throughout the length of the filter.

Typically the largest group of pores in the distribution of openings will have a maximum average cross sectional dimension of no more than about 180μ, no more than about 130μ, no more than about 110μ or 100μ or 80μ or less, discounting larger potential statistical outliers that have no meaningful effect on performance. The membrane may be configured to block the passage of debris as small as 0.5 mm and greater, or 0.25 mm and greater, or 0.1 mm and greater or less.

Electrospinning refers generally to processes involving the expulsion of flowable material from one or more orifices under a high voltage electric field, and the material forming fibers are subsequently deposited on a collector. Examples of flowable materials include dispersions, solutions, suspensions, liquids, molten or semi-molten material, and other fluid or semi-fluid materials.

In some instances, the rotational spinning processes are completed in the absence of an electric field. For example, electrospinning can include loading a polymer solution or dispersion, including any of the cover materials described herein, into a cup or spinneret configured with one or more orifices on the outside circumference of the spinneret. The spinneret is then rotated (or the wire frame rotated near a fixed spinneret), causing (through a combination of centrifugal and hydrostatic forces, for example) the flowable material to be expelled from the orifices. The material may then form a “jet” or “stream” extending from the orifice, with drag forces tending to cause the stream of material to elongate into a small diameter fiber. The fibers may then be deposited on the wire frame.

Through a series of passes of the spinneret axially and with rotation relative to the frame, the fibers can be ‘layered’ along the frame effectively successively reducing the average pore sizes formed between adjacent fibers. Further information regarding electrospinning can be found in U.S. Publication No. 2013/0190856, filed Mar. 13, 2013, and U.S. Publication No. 2013/0184810, filed Jan. 15, 2013, which are hereby incorporated herein by reference in their entirety.

In addition to pore size distribution, open area, thickness of the covering and membrane flexibility all may affect filter performance.

As the frame is deployed around a curve, a pore on the outer edge elongates in length in the direction of the curve and is compressed perpendicular to the length. This can be up to 50% increase in length while the perpendicular measurement appears to decrease by about 40%.

For a pore on the concave surface of the curve, the change in length along the curve is less obvious, it generally decreases in length about 3% under compression while the perpendicular measurement remains essentially unchanged. As the filter elongates, the porosity and pore size is reduced. Porosity at very small aorta diameters (22 mm diameter) is approximately 0.20 (20 percent).

The filter pore size preferably substantially maintains filter efficiency throughout these range of conditions. As measured by the porosity and observationally of large pore dimensional behavior, the maximum pore size is defined by the coating process at the fully deployed diameter. Elongation of the frame reduces the diameter, porosity and maximum size of particle that will fit thru the pore.

The filter preferably can perform at multiple vessel diameters throughout the range of from about 22 mm to about 45 mm and over the aortic arch which can have multiple radii and three dimensional tortuosity. The covering adds very little to the device profile (about 0.0005″) and minimally affects the flow characteristics to the covered blood vessels. The filter cover appears to be adherent to the frame, durable and not thrombogenic. Testing has demonstrated porosity ranges from about 40% at fully deployed diameter of about 31.6 mm, down to about 25% porosity at about 22 mm diameter.

For the effect of decreasing diameter on porosity (% open space), porosity climbs as diameter is increased:

| Frame Diameter | % Open Area |
|---|---|
| 32 mm (as built) | 41.8% |
| 28 mm (straight) | 33.0% |
| 24 mm (straight) | 27.8% |
| 20 mm (straight) | 19.7% |

This may be attributable to the pores axially elongating and narrowing circumferentially as diameter goes down.

This pore size distribution creates multiple levels of filtering, created by the multiple average pore size groups in the filter (e.g., 80μ and below, 60μ and below, 40μ and below, etc.). A first group of pores may have the largest average diameter, such as about 80 μm or less. A second group of pores may have a smaller average diameter, such as about 60 μm or less. A third group of pores may have a smaller average diameter, such as about 40 μm or less.

In terms of total pore count, the third group will have the most pores followed by the second group, and the first group will have the fewest number of pores. This optimizes perfusion, while blocking particles of a size sufficient to independently create an adverse risk, while also blocking passage of a number of particles below the threshold of the first group, having the consequence of reducing the total delivered embolic load. This should lead to less total volume of particles passing through the filter to the brain compared to conventional filters which only block particles above a single threshold size, but do not reduce the cumulative volume of particles below that threshold from reaching the brain. It is recognized in the art that the total volume of debris to the brain, correlates to cognitive impairment & stroke. This additional filtering is achieved with no clinically significant reduction on blood flow to the brain, and in some implementations no measurable drop in pressure across the membrane.

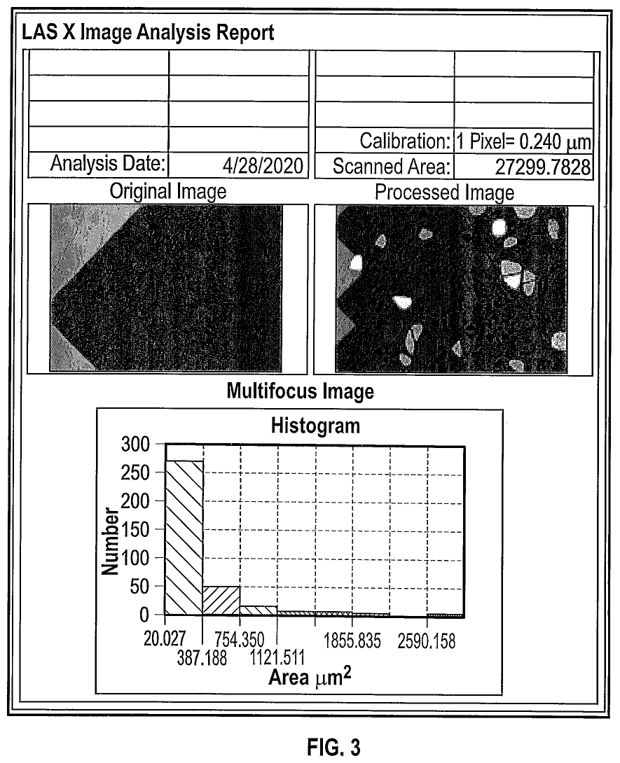

FIG. 3 is an image and analysis performed with a Leica microscope and Leica image analysis software, to map the pore size distribution. By far the greatest number of pores (first row in the chart) is a first group of pores within the 20-387 square microns size range (equivalent to as large as a 22 micron diameter pore). The next most predominant number of pores is a second group of pores within the 387 to 754 square microns size (31 micron). A third group of pores is within the 754 to 1122 square micron size (38 micron). The largest observed hole is nominally 3000 square microns (equivalent to a 61.8 micron diameter pore). Additional pore distribution data is shown in the table below:

| | Area [μm²] | Area [μm²] | | Percent of |
|---|---|---|---|---|
| Bin | Lower | Upper | Count | Total Number |
| Bin 1 | 20.027 | 387.188 | 259 | 78.198 |
| Bin 2 | 387.188 | 754.350 | 48 | 13.953 |
| Bin 3 | 754.350 | 1121.511 | 13 | 3.779 |
| Bin 4 | 1121.511 | 1488.673 | 6 | 1.744 |
| Bin 5 | 1488.673 | 1855.835 | 3 | 0.872 |
| Bin 6 | 1855.835 | 2222.996 | 2 | 0.581 |
| Bin 7 | 2222.996 | 2590.153 | 0 | 0.000 |
| Bin 8 | 2590.158 | 2957.319 | 3 | 0.872 |

While the number count of pores is driven by smaller pores, a small process change can increase the number of 25-30 micron pores. This allows the average pores size to increase slightly and raises the average pore size. Preferably, the histogram will reveal a normal distribution around 30-40 microns which provides good porosity and minimize pressure drop while still having a great particle protection feature.

As the blood and any particles hit the filter, if the particle is smaller than the pore size it happens to encounter, it can pass through the filter. If it is larger than the pore it encounters, it is stopped at the surface of the filter. Arterial flow through the central lumen of the filter will deflect much of the particle mass stopped at the surface of the filter downstream, away from the cerebral vessel, and minimize the risk of the filter becoming occluded and raising a pressure drop across the filter.

In one implementation the filter can thus filter all particles greater than a preset threshold (e.g., 80 microns) but it will also provide some filtration of particles of lower sizes as shown in the histogram of FIG. 3 due to the randomness of particle size and pore size encounters. At a certain pore size (8-10 microns it essentially filters and prevents red blood cells from passing. But the number of pores of that small size are insignificant so essentially all pores allow the passage of blood and the large total open area (sum of all pores) is sufficient for the filter to impose no clinically meaningful pressure drop across the filter.

Preferably, the filter will exhibit a relatively low radial force throughout a wide range of expanded diameters. This results from the use of small diameter wire, and the total radially outwardly facing surface area of the wires being a relatively low percentage of the surface area of the filter.

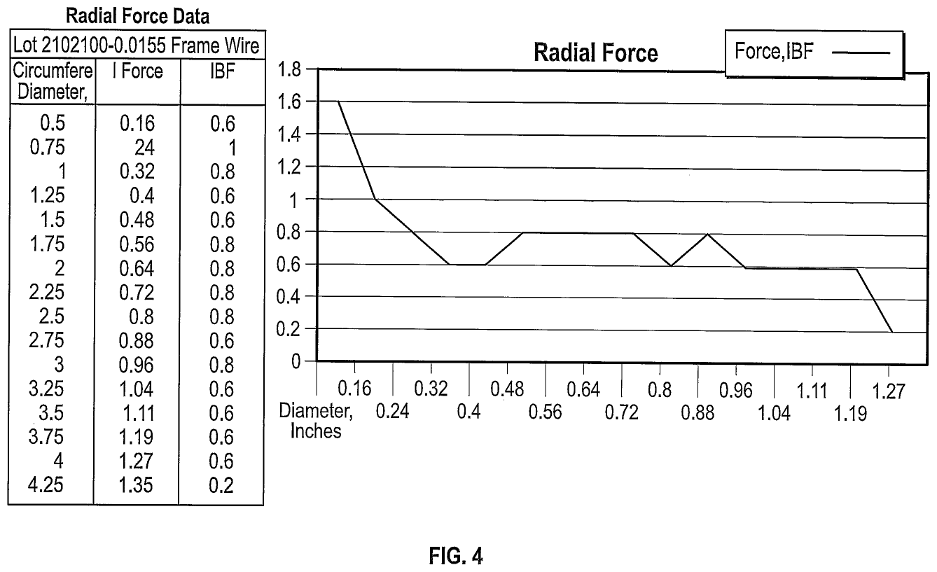

In one example, as demonstrated in FIG. 4, the filter has a radial force of no more than about 1.4 lbf, preferably no more than about 1.0 and in the illustrated example, no more than about 0.8 lbf at an expanded diameter of about 0.24 inches. Radial force is maintained within the range of from about 0.4 lbf to about 1.0 lbf all the way up to an expanded diameter of at least about 1 inch.

FIG. 5A shows a closed distal, downstream end filter to be placed from the cannula or transapically that is designed to be placed in the ascending aorta to filter blood entering through the perfusion cannula and have all blood passing through the aorta filtered through the membrane. The length of the cylindrical portion of the filter is designed to accommodate varied diameters of aorta and may be 2 cm long in a 30-32 mm diameter aorta and 4 cm long in a smaller aorta of approximately 25 mm diameter.

FIG. 5B shows a closed end filter to be placed thru the Left subclavian artery. The end of the filter would be placed between the R-innominate artery and the cross-clamp location. An elongate transition 124 may have an axial length of at least about 25%, in some implementations at least about 40% or 50%, or more of the overall length of the device excluding control wire 42.

The filter illustrated in FIG. 5C would be placed either from the L-Subclavian, or from the groin. Due to the short tapered area 124 the distal end of the device would be placed between the R-innominate artery and the cross-clamp location. The entire cylindrical portion of the filter would reside in the aortic arch. The axial length of the transition zone 124 may be no more than about 20%, or no more than about 15% or 10% of the axial length of the device excluding control wire 42

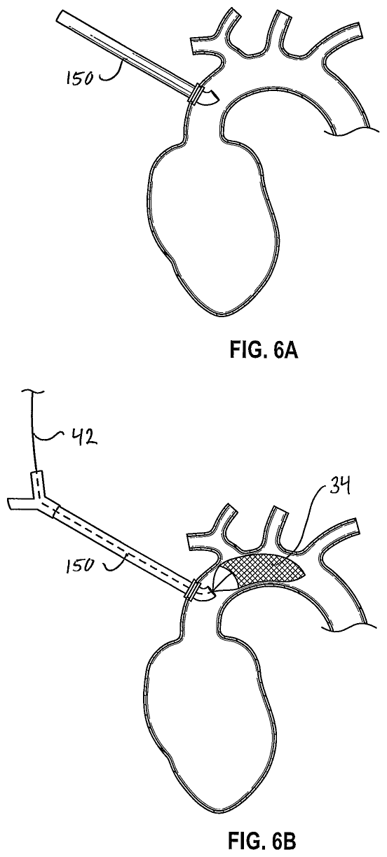

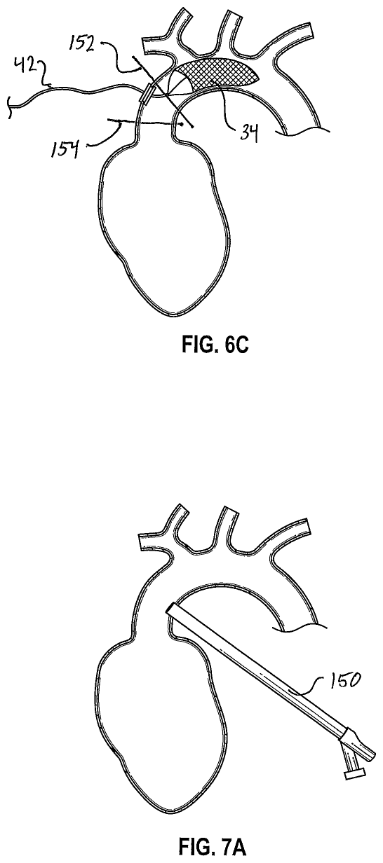

FIGS. 6A-6C show direct placement of the filter during open cardiac surgery, via cannula 150. Following placement of the filter, the cross clamp may be applied along a first cross-clamp plane 152, over the control wire 42. Alternatively, the cross-clamp may be applied across a second cross-clamp plane 154. In some embodiments, the cross-clamp may be applied across a portion of the aorta containing the filter itself, such as the filter frame 34. The filter may be in the location shown in FIG. 6C, or it may be longer and/or located farther proximally, such that the cross-clamp plane 152 extends over the filter frame 34 portion of the filter, and the cross-clamp is applied across the filter frame 34. In some embodiments, the filter frame 34 may include nitinol wire having a diameter that is no greater than about 0.009″, such as 0.009″, 0.008″, 0.007″, or 0.006″, and the cross-clamp may be applied across the region of the aorta containing such filter. Such embodiments may reduce the risk of damage to the aorta as compared to filters with large diameter wire frames, while still providing sufficient resilience and robustness to self-expand after removal of the cross-clamp, and such that the filter still performs optimally. Further, by clamping over the filter, such technique may secure the filter in place during the procedure. Any of the embodiments or methods described herein may incorporate these features of cross-clamping over the filter.

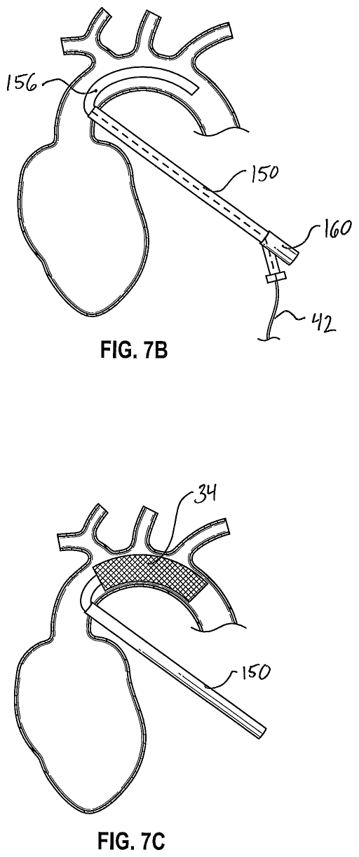

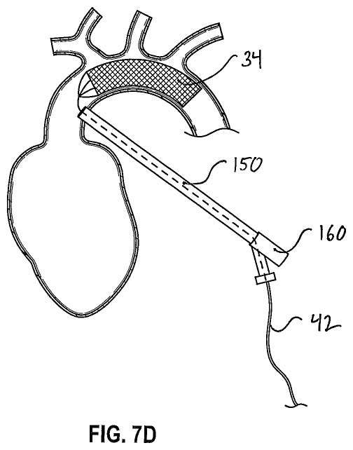

Referring to FIGS. 7A-7E, the filter may be deployed through the aortic cannula hole. The filter is deployed after the holes is cut and deployed distal to the left subclavian artery and extending over the arch and anchoring in a grommet used to hold the aortic cannula used during bypass.

Referring to FIGS. 7A-7D, the filter may alternatively be deployed through the arterial cannula. The aorta is accessed with a cannula 150 having a Y fitting 160 with a hemostatic valve between the arterial cannula and the tubing set to allow access to the aorta. The deployment catheter 156 may thereafter be introduced through the hemostatic valve and via the cannula 150 into the aorta. The deployment catheter 156 may thereafter be proximally withdrawn to expose the filter 34, allowing it to expand across the aortic arch. The deployment catheter 156 may thereafter be proximally withdrawn, leaving the control wire 42 extending through the cannula and access port. As with other implementations disclosed herein, the aorta may be clamped over the control wire 42, so that the filter can remain in place during the index procedure and then be retrieved proximally following removal of the cross clamp.

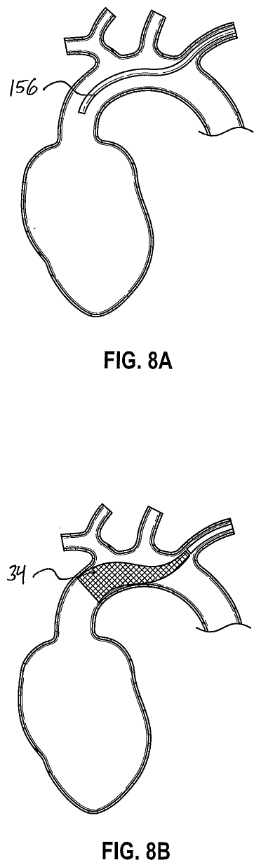

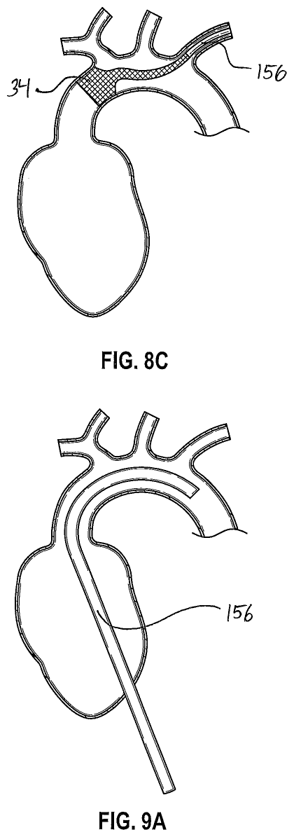

Referring to FIGS. 8A-8E, the filter is designed to enter from the left radial artery or left subclavian artery, and is deployed within the aorta covering the innominate artery, left carotid artery and potentially entering the left subclavian artery, providing embolic protection to all three great vessels. The arterial cannula penetrates the aorta wall and delivers oxygenated blood to the aorta, the brain and the distal vasculature during the procedure.

The filter deployment catheter 156 may include an outer sheath, that contains the collapsed filter, a pushrod to support the filter during deployment and retrieval, and a handle to allow manipulation of the filter and catheter for deployment and retrieval. The sheath may be braid or coil reinforced to support the lumen and prevent kinking thru any curvature. This will allow manipulation with, or without a guidewire. The delivery catheter will be soft and pre-shaped to allow delivery with ultrasound guidance.

Sequence of use for left subclavian deployment:

1) Access the vasculature via the left subclavian artery with a sheath/introducer.

2) Introduce the catheter and filter thru the sheath and into the subclavian artery and advance into and over the aortic arch.

3) Monitor position of the distal end of the catheter with transesophageal echocardiography. Place the distal end of the catheter approximately 1-2 cm distal to the innominate artery.

4) Deploy the filter—by slowly withdrawing the sheath while holding the handle in place via “pin and pull” technique. Monitor deployment of the filter with the echocardiography.

The filter terminates proximally in the left subclavian artery with the delivery catheter acting as a funnel to close the filter.

5) Once the filter is placed and conforming to the aorta walls, the surgical procedure can proceed following cross-clamping of the aorta, approximately 0.5-2 cm distal to the end of the filter, and introduction of the arterial cannula for perfusion.

6) When the surgical procedure is completed, the heart and aorta refilled with blood, the cross clamp can be removed. Any particles liberated during this procedural step are collected in the filter and prevented from embolizing the cerebral vasculature.

7) The filter can then be recaptured in the catheter by holding the handle and advancing the outer sheath over the filter.

8) Finally, the catheter can be withdrawn from the aorta and vasculature and the access sheath removed.

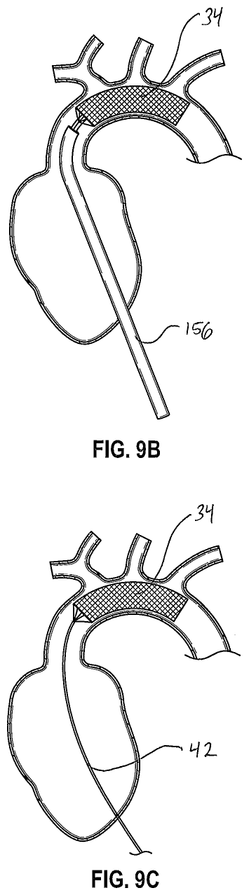

For the trans-apically accessed filter, an apical access port allows passage of the device thru the heart wall and left ventricle, passing thru the aortic valve and over the aortic arch. See FIGS. 9A-9C. The filter is then deployed initially expanding in the aortic arch distal to the left subclavian artery orgin. It then expands as the sheath is withdrawn over the aortic arch and is finally deployed leaving a retrieval wire 42 in the ascending aorta. This can be cross clamped when the aortic valve replacement starts.

To maintain a soft filter that can be deployed and also minimize the diameter of the delivery device 0.004″ to 0.012″ nitinol superelastic wire is anticipated to be braided, heat treated and the coated with the filter membrane. In some embodiments, the wire may be 0.006″ or 0.009″. Larger diameter wire can be used and would be suitable although it will generally increase the diameter of the delivery device. Other suitable wire would include cobalt-chrome wire which allows for higher retention forces at smaller wire diameters due to its substantially higher modulus.

What is claimed is:

1. An intravascular filter for blocking passage of selected sizes of debris, comprising:
a self expandable tubular wire frame, having a first end, a second end and a tubular sidewall defining a central lumen;
a tubular porous membrane carried by the sidewall, the membrane having a distribution of pore sizes;
wherein a first group of pores has pores with a maximum dimension of no more than about 25 microns and a second group of pores has pores with a maximum dimension of at least about 50 microns, and the prevalence of pores in the first group is at least three times the prevalence of pores in the second group; and
a porous membrane extending across the second end, so that debris entering the first end can be captured within the central lumen.

2. An intravascular filter as in claim 1, wherein the second group of pores will block particles greater than about 120 microns.

3. An intravascular filter as in claim 2, wherein the second group of pores will block particles greater than about 100 microns.

4. An intravascular filter as in claim 3, wherein the second group of pores will block particles greater than about 80 microns.

5. An intravascular filter as in claim 1, wherein the prevalence of pores in the first group is at least four times the prevalence of pores in the second group.

6. An intravascular filter as in claim 1, wherein a pressure drop across the filter is less than about 10 mm Hg at physiologic flow rates.

7. An intravascular filter as in claim 6, wherein a pressure drop across the filter is less than about 5 mm Hg at physiologic flow rates.

8. An intravascular filter as in claim 1, wherein the sum of the area of all of the pores is at least about 30% of the surface area of the membrane.

9. An intravascular filter as in claim 8, wherein the sum of the area of all of the pores is at least about 35% of the surface area of the membrane.

10. An intravascular filter as in claim 1, wherein the tubular porous membrane comprises electrospun fibers.

11. An embolic protection system, comprising:
a self expandable frame having a proximal end and a distal end;
a filter membrane supported by the tubular body, surrounding the tubular body and covering the distal end; and
a transition between the frame and a control wire extending proximally from the transition;
wherein the transition comprises a first set of wires extending proximally from the frame to a first set of welds, and a second, smaller set of wires extending proximally from the welds, and the welds are axially displaced from each other.

12. An embolic protection system as in claim 11, further comprising a tubular delivery catheter, and the frame is carried in a reduced cross-sectional configuration within the delivery catheter.

13. An embolic protection access system as in claim 12, wherein the delivery catheter has an outer diameter of less than 14 F.

14. An embolic protection system as in claim 11, wherein the filter membrane comprises electrospun fibers.

15. An embolic protection system as in claim 11, wherein the filter membrane has a distribution of pore sizes, wherein a first group of pores has pores with a maximum dimension of no more than about 25 microns and a second group of pores has pores with a maximum dimension of at least about 50 microns, and the prevalence of pores in the first group is at least three times the prevalence of pores in the second group.

16. A method of protecting the cerebral vasculature from embolic debris, comprising:
providing an embolic protection delivery catheter having a tubular restraint for restraining an embolic protection filter in a reduced profile configuration, the filter having a self expandable wire frame, a filter membrane carried by the frame and covering at least an end of the frame, and a proximal and distal radiopaque markers;
advancing the filter distally through an access sheath into the aorta;
retracting the restraint to expose the filter and permit the frame to radially expand, leaving a control wire extending proximally from the filter; and
cross clamping the aorta.

17. The method of claim 16, wherein the cross clamping step comprises cross clamping the aorta over the embolic protection filter.

18. The method of claim 16, wherein the cross clamping step comprises cross clamping the aorta over the control wire.

19. The method of claim 16, further comprising removing the cross clamp from the aorta and allowing blood to perfuse through the membrane in the direction of the descending aorta while retaining embolic debris therein for subsequent removal.

20. The method of claim 16, wherein the embolic protection delivery catheter is delivered to the aorta via i) direct placement of the filter during open cardiac surgery, ii) through an aortic cannula hole, iii) through a left radial artery or left subclavian artery, or iv) trans-apically.
